(19) United States
(12) Patent Application Publication
(10) Pub. No.: US 2009/0187121 A1
(43) Pub. Date: Jul. 23, 2009

(54) DIARY AND METHOD FOR MEDICAL MONITORING

(75) Inventor: Tom Evans, Cambridgeshire (GB)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: CamNtech Limited, Cambridge (GB)

(21) Appl. No.: 12/271,387

(22) Filed: Nov. 14, 2008

(30) Foreign Application Priority Data
Jan. 22, 2008 (GB) GB 0801145.0

(51) Int. Cl. A61B 5/11; A61B 5/00

(52) U.S. Cl. 600/595; 600/300

(57) ABSTRACT

A medical-monitoring diary device comprising a housing, a display supported by the housing, a wrist strap extending from the housing such that the diary can be worn on a user's wrist, a processor housed within the housing coupled to the display, and a memory coupled to the processor in which the diary comprises at least one slider and one input button coupled to the processor for receiving inputs from the user, for storage in the memory, and the processor controls the display for displaying messages to be viewed by the user.

### FIELD OF THE INVENTION

Embodiments of the invention relate to a diary device and a method for medical monitoring and, in particular, to a wrist-worn diary for medical monitoring.

### BACKGROUND OF THE INVENTION

For users undergoing medical monitoring, keeping a record of aspects of their condition and behaviour may be desired. This may include or involve monitoring factors such as sleep quality or pain. This may for example be in a medical treatment context, involving the treatment of patients, or in a research or development context, involving monitoring volunteer subjects in a clinical trial.

Conventionally, paper diaries can be used for medical monitoring. Paper diaries are cheap and are sometimes well used, but are often either not filled in at all, or filled in retrospectively from memory, resulting in poor data quality.

General-purpose personal digital assistants with custom diary software can be used for data input during medical monitoring, but are not well adapted to the task. The general purpose PDA deliberately contains a relatively large screen to maximize its usefulness. This prevents it from being worn continually on the wrist or other part of the body. In the daily life of a subject or user, particularly when undergoing long-term monitoring, the PDA diary is often not on hand when an entry needs to be made. In addition, the units are often lost or stolen due to their general usefulness for other purposes.

In practice, it may be desirable for a medical-monitoring diary to be conveniently on hand whenever an input from a user is required, and that it is preferably very simple and easy to use. This is because a user using such a diary often has limited ability to use such a device. For example, a person in need of medical monitoring and provided with a medical-monitoring diary, or patient diary, may well be elderly or suffering from a serious medical condition that may cause significant pain or loss of motor function, that makes it difficult to operate, or even to remember to operate, a medical-monitoring diary.

For these reasons, it may be desirable for the diary to be as convenient as possible to use and that the interface between the user and the diary be as clear and simple as possible, while retaining the ability to acquire from a user the information required by a physician to monitor their medical condition.

The inventors of the present invention have previously developed similar devices, as described for example in U.S. Pat. No. 6,129,663 and in US Patent Application No. US-2005-00228698-A1. Both of these devices use simple, user-friendly interfaces to acquire information from users.

Actigraphy units (for monitoring a user's movement) with marker or scoring buttons can also be worn on the wrist, but only allow very simple answers to be recorded. For example, a conventional actigraphy unit may continuously monitor a user's physical movement, or activity, and comprise a button for the user to press when going to bed or when waking up. The unit then marks these times on the activity record.

### SUMMARY OF THE INVENTION

Certain embodiments of the invention may thus advantageously provide a medical monitor, or medical monitoring diary, that may be wrist-worn and which has a display viewable or readable by the user, and a slider and an input button for use by the user to input information to the diary. The slider and the button may be mechanical components or may be touch sensors, such as capacitive or resistive touch sensors, or may comprise any similar arrangement for detecting the user's finger. The slider and the button need not be implemented in the same way; for example, the slider may be a touch sensor and the button a mechanical push-button, or vice versa.

The display may be a touch-sensitive display and, in that case, may be used to implement the slider and/or the button.

In some embodiments, only a single slider and a single button may be provided, to achieve simplicity of operation.

The display may show messages viewable by the user, either in the form of text or images or in any other way. Such messages may request information from the user, to be input using the slider and/or the button, or may give feedback to the user, for example following an input of information.

In some embodiments, the diary device comprises a memory for storing information input by the user. It may also allow the storage of messages for display to the user. These aspects could be stored using separate memories if desired. The processor may be programmed with any predetermined messages and to receive any desired user input in response to those messages.

In some embodiments, the diary device includes a clock so that the diary can record the times at which the user inputs information. The diary device may also be programmed with an operating schedule, for example concerning times at which a medication should be taken, or times at which the diary device may prompt the user for an input regarding their condition.

### BRIEF DESCRIPTION OF THE DRAWINGS

The following drawings are illustrative of particular embodiments of the invention and therefore do not limit the scope of the invention. The drawings are not necessarily to scale (unless so stated) and are intended for use in conjunction with the explanations in the following detailed description. Embodiments of the invention will hereinafter be described in conjunction with the appended drawings, wherein like numerals denote like elements.

FIG. 1 is a perspective view of a medical-monitoring diary according to a first embodiment of the invention;

FIG. 2 is a perspective view of a medical-monitoring diary according to a second embodiment of the invention; and

FIGS. 3 to 10 illustrate the display of information on a screen of the medical monitoring diary according to the second embodiment, and input of data by the user.

### DETAILED DESCRIPTION

The following detailed description is exemplary in nature and is not intended to limit the scope, applicability, or configuration of the invention in any way. Rather, the following description provides practical illustrations for implementing exemplary embodiments of the invention.

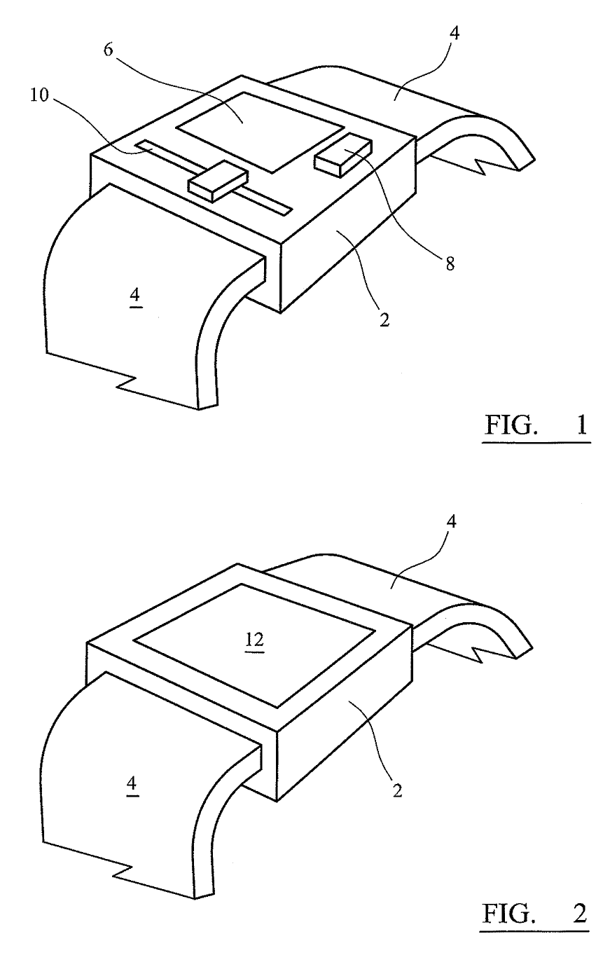

FIG. 1 illustrates a medical-monitoring diary according to a first embodiment of the invention. The diary comprises a housing 2 mounted on a wrist strap 4 (shown only in part) so that it may conveniently be worn on the user's wrist. A display 6, such as a liquid crystal display, is mounted on a front face of the housing 2, so as to be viewable by the user. A push button 8 is mounted alongside the display and a slider 10 is mounted below the display, for use by the user to input information to the diary.

Within the housing 2, a processor is coupled to the display for displaying messages to be viewed by the user, and to the button and the slider for receiving inputs from the user. The processor is coupled to a memory, also housed within the housing, for storing information input by the user and for storing the messages. The housing 2 also contains a clock, which the processor may use to generate messages at predetermined times, and to record the times at which information is input by the user.

The housing 2 also contains a transducer, coupled to the processor, for producing audible alarms for alerting the user. This may also be usable for generating voice messages to the user, particularly if the user has difficulty in viewing messages displayed on the display.

The housing 2 also contains a battery for powering the diary.

The processor is connectable to an external computer, either through a wireless link or a wired linked (not shown) for downloading stored information and/or for programming the processor for future operation.

FIG. 2 shows a preferred embodiment of the invention comprising a housing 2 and a wrist strap 4 similar to those in FIG. 1, but in which a touch-sensitive display 12 is mounted on the front face of the housing 2. The touch-sensitive display 12 may advantageously be larger than the display 6 in the embodiment of FIG. 1 and the slider and push-button of FIG. 1 may be implemented as corresponding regions of the touch-sensitive display, under the control of the processor.

Operation of the Medical-Monitoring Diary

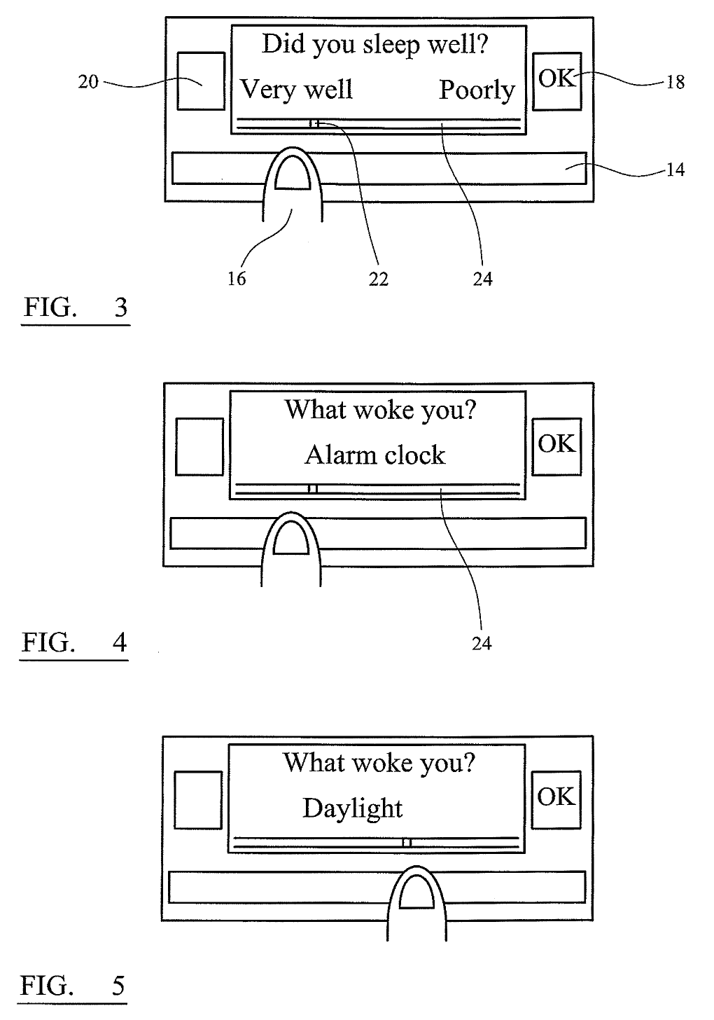

When the user wakes up in the morning, they wake the diary by pressing the button 8 (in the embodiment of FIG. 1) or touching the touch-sensitive display 12 (in the embodiment of FIG. 2). The following description will use the embodiment of FIG. 2 by way of example. The processor then controls the display 12 to display the message ‘Did you sleep well?’, as illustrated in FIG. 3. A slider 14 is implemented on the touch-screen and the user indicates with their finger 16 the answer to the question ‘Did you sleep well?’. This information is input as a continuously-variable parameter between the extreme values ‘Very well’ and ‘Poorly’. This is displayed by the display as a mark 22 on an icon 24 representing the slider. When the user has indicated an appropriate value on the slider, they press a button 18 marked ‘OK’, also implemented on the touch screen, to confirm their input. The display also shows a second touch-sensitive button 20, which is not used at this stage and could be omitted if desired.

In the embodiment, the diary also records in its memory the current time, indicating when the user woke.

The diary then displays a second question, ‘What woke you?’, as illustrated in FIGS. 4 and 5. This is a multiple-choice question and as the user moves their finger across the slider, different predetermined answers are displayed. Two example finger positions are shown in FIGS. 4 and 5, corresponding the answers ‘Alarm clock’ and ‘Daylight’. An icon 24 on the screen again displays the position of the user's finger on the shield. When the user has selected their preferred answer, they again press the ‘OK’ button 18.

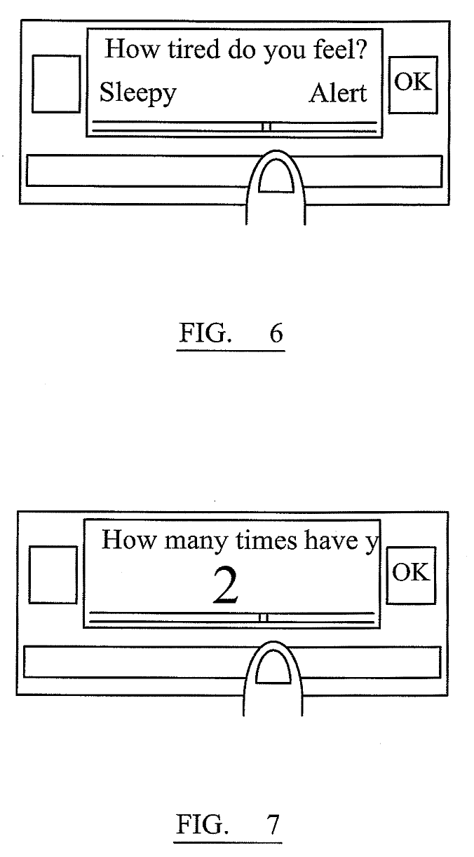

After the user has answered these questions after waking, the unit displays a thank-you message before returning to a low-power mode. It is then programmed to wake at certain times during the day and asked other questions, as illustrated in FIGS. 6 to 9. The timing of these questions or messages is predetermined by the programming of the processor and depends on the aspects of the user's condition that are to be monitored.

When the diary requires an input of this type, it attracts the user's attention by means of a buzzer signal or a flashing signal. Examples of questions that may be displayed are shown in FIGS. 6, 7 and 9. FIG. 6 illustrates the question ‘How tired do you feel?’. This requires an input on a continuously-variable scale between the extreme values ‘Sleep’ and ‘Alert’. The user selects an appropriate value using the slider and presses the ‘OK’ button. Depending on the answer to the question, the unit may ask a further question, such as illustrated in FIG. 7. This question is ‘How many times have you nearly fallen asleep today?’. This question is continually scrolled across the display as it will not fit within a single screen width. The answer to this question is an integer number. In order to select the number, the following process is used. An initial value, preferably ‘0,’ is displayed on the screen and the user can change the number by repeatedly dragging the slider left or right. For example, moving the user's finger from left to right across the slider increases the displayed value from ‘0’ to ‘1’. Again dragging the slider to the right increases the value to ‘2’ as shown in FIG. 7. Dragging the slider across its travel from right to left decreases the displayed integer by one. When an appropriate value is displayed on the screen, the user presses the ‘OK’ button to confirm the input.

This method of inputting information such as integer numbers may advantageously be performed by repeated operation of the slider across its full width, or a significant portion of its width, particularly if the user suffers from limited or impaired motor function. The wrist-worn device is relatively small, and small devices are potentially difficult to operate for those with impaired motor function or visual function. Consequently, it may be preferred that the medical-monitoring diary of the embodiment be as easy as possible to use.

It should be noted that this technique of repeated operation of the slider across its whole length, or a substantial portion of its length, may be used for inputting any parameter that is not continuously variable, such as the integer number illustrated in FIG. 7 or the answer to a multi-choice question as illustrated in FIGS. 4 and 5. Rather than the finger position on the slider corresponding to different answers as described in relation to FIGS. 4 and 5 above, a full sweep of the finger across the slider may be used to change the answer displayed on the screen from one multiple-choice answer to another.

This principle may be extended to the answering of questions having continuously-variable answers, such as the questions illustrated in FIGS. 3 and 6. In that case, more than one repeated operation of the slider may be required to move the position of the slider indicated on the display between the extreme values of the parameter shown on the display.

During operation, a further button 20 may be displayed on the screen and may be used to skip or go back questions if desired. Additionally, when no question is being asked, the button 20 may be used to access a system menu for such tasks as selecting a silent, non-prompting mode, checking the time of the next question set, or viewing and adjusting other basic configuration options.

In practice, it may be desirable to omit the button 20 from the display while the medical-monitoring diary is being used by a user being monitored, in order to simplify the operation of the diary as far as possible.

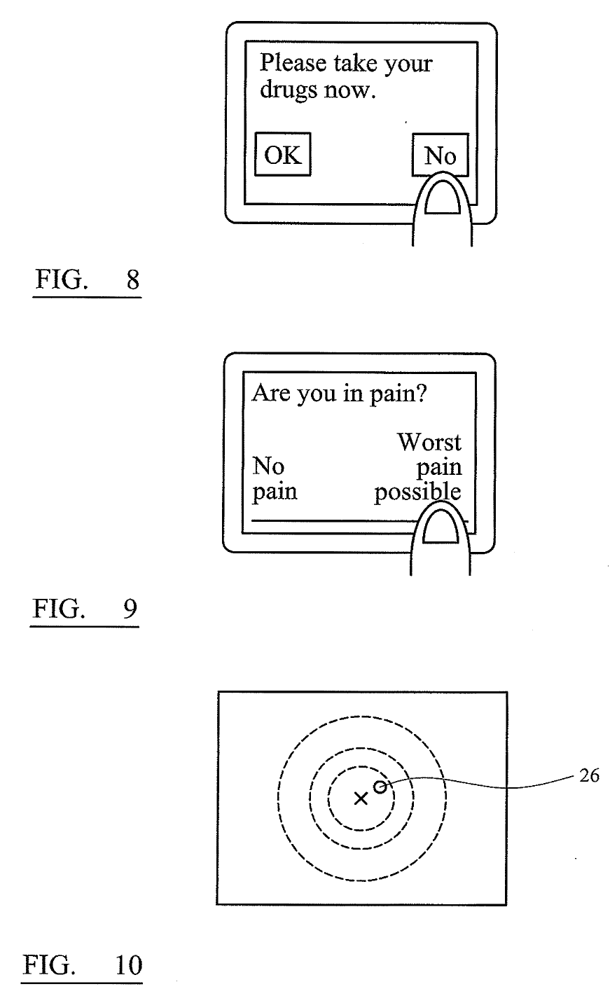

The diary can also be used for monitoring drug use. In this case, the diary prompts the user with an audible noise or flashing light or display at a predetermined time. After their attention is gained, it may display a message as shown in FIG. 8 ‘Please take your drugs now’. In this case, no slider is displayed, but only two buttons marked ‘OK’ and ‘No’. If the user takes their drugs as prescribed, they should indicate ‘OK’. If they do not take their drugs at this time, they should press the button ‘No’. If the user presses the ‘No’ button, then the diary may ask further questions to ascertain the reason for non-compliance.

Following the drug-taking prompt, the diary may question the user about their condition, for example monitoring pain management as illustrated in FIG. 9. Here, the user is asked the question ‘Are you in pain?’ and is asked to input a continuously-variable value between ‘No pain’ and ‘Worst pain possible’. In FIG. 9, the user is indicating ‘Worst pain possible’.

In FIG. 9, the touch screen is configured in an alternative way to the screen as illustrated in FIGS. 3 to 7, by way of example. In this case, the user uses their finger to indicate a value directly on a slider, and no slider-position indicator is displayed (or is displayed beneath the user's finger) during the information input.

The diary may ask further questions, depending on the answer to the question ‘Are you in pain?’, or could suggest contacting a doctor, even displaying a contact phone number or web address for further advice.

In a further embodiment, the diary may implement a reaction time test. This may require the user to press a button or tap a touch-screen in response to a signal displayed on the display, and so that the diary can measure their reaction time and/or accuracy. This test may be implemented as a component of a monitoring protocol in order to quantify objectively the effect of drugs or other treatment.

The medical-monitoring diary may further comprise an accelerometer, mounted within the housing and coupled to the processor. This may be used as part of a test for reaction time, or may be used in other tests as illustrated in FIG. 10. In this case, a marker 26, representing a ball, is displayed on the screen, together with a series of fixed concentric rings. The image of the ball is movable on the display screen, as if it were rolling across the screen. The user is asked to ‘balance’ the ball in the display, keeping it centred within the concentric rings. The user's success in this task may be monitored and the accelerometer may be used to monitor their movements during the test, for examples for signs of tremor. Again, this may be used to quantify objectively the effect of drugs or other treatment, as well as monitoring the user's condition.

Where the diary incorporates an accelerometer, movement monitoring may be performed continuously, as in a conventional wrist-worn actigraphy device.

In the foregoing detailed description, the invention has been described with reference to specific embodiments. However, it may be appreciated that various modifications and changes can be made without departing from the scope of the invention as set forth in the appended claims.

1. A medical-monitoring diary device, comprising;
a housing;
a display supported by the housing;
a wrist strap extending from the housing such that the diary device can be worn on a user's wrist;
a processor housed within the housing and coupled to the display; and
a memory coupled to the processor,
wherein the diary device comprises at least one slider and one input button coupled to the processor for receiving inputs from the user for storage in the memory, and the processor controls the display for displaying messages to be viewed by the user.

2. The device of claim 1, wherein the display is a touch-sensitive display and the processor controls the display to implement the slider.

3. The device of claim 1, wherein the display is a touch-sensitive display and the processor controls the display to implement the one input button.

4. The device of claim 1, further comprising a clock coupled to the processor.

5. The device of claim 1, further comprising a motion sensor coupled to the processor, for example for monitoring the user's movement or activity.

6. The device of claim 5, wherein the diary device implements a test for tremor or other aspect of the user's movement.

7. The device of claim 6, wherein a marker or icon is displayed on the display, and the user is required to control movement of the marker or icon on the display through movement or tilting of the diary device.

8. The device of claim 1, further comprising a transducer coupled to the processor for generating an audible signal.

9. The device of claim 1, wherein the slider is usable either to input a user estimate of a continuously variable parameter or to select between a plurality of discrete options, for example in response to a question displayed on the display.

10. The device of claim 1, wherein the user wakes the device when the user wakes in the morning, and inputs an estimate of their sleep quality during the night using the slider.

11. The device of claim 1, wherein the slider is usable to input an integer selected by the user, an integer being displayed on the display and the user repeatedly sliding their finger along the slider to increment or decrement the integer by one each time.

12. The device of claim 1, wherein the diary device may be programmed to generate a visible and/or audible and/or vibratory alarm at a predetermined time or times.

13. The device of claim 12, wherein an alarm is generated at a time when the user is to take a medication, or perform an action, the diary device displays a question to the user, and invites the user to provide an input to indicate whether or not the user has taken the medication or completed the action.

14. The device of claim 12, wherein an alarm is generated at one or more predetermined times and displays a question requesting from the user an input corresponding to an aspect of their condition, such as an indication of their current alertness or their current pain.

15. The device of claim 1, wherein a message or question for the user is generated as an audio prompt, in addition to or instead of being displayed on the screen.

16. The device of claim 1, wherein the display displays a message and requires the user to provide an input in order to test the user's reaction time.

17. A method for medical monitoring comprising;
proving a wrist-worn diary to a user, the diary having a slider and a button for receiving inputs from the user;
displaying on a display of the diary a message to the user requesting input;
providing an input to the diary using the slider; and
storing the input in a memory of the diary.
